(19) United States
(12) Patent Application Publication
(10) Pub. No.: US 2016/0278679 A1
(43) Pub. Date: Sep. 29, 2016

(54) MEDICAL DEVICES AND INSERTION SYSTEMS AND METHODS

(71) Applicant: ABBOTT DIABETES CARE INC., Alameda, CA (US)

(72) Inventors: R. David Woodruff, Alameda, CA (US); Mohammed E. Moein, Saratoga, CA (US); Phillip Yee, San Francisco, CA (US)

(21) Appl. No.: 15/158,270

(22) Filed: May 18, 2016

Related U.S. Application Data
(63) Continuation of application No. 13/171,388, filed on Jun. 28, 2011, now abandoned.
(60) Provisional application No. 61/359,815, filed on Jun. 29, 2010.

(51) Int. Cl. A61B 5/1486; A61B 5/1495; A61B 5/00; A61B 5/145

(52) U.S. Cl. CPC A61B 5/14865; A61B 5/14532; A61B 5/1495; A61B 5/7282

(57) ABSTRACT

Implantable medical devices, systems, methods and kits for transcutaneous insertion of the implantable medical devices are provided.

### RELATED APPLICATIONS

The present application is a continuation of U.S. patent application Ser. No. 13/171,388, filed Jun. 28, 2011, which claims the benefit of U.S. Provisional Application No. 61/359,815, filed Jun. 29, 2010, entitled “Medical Devices and Insertion Systems and Methods”, both of which are incorporated herein by reference in their entireties for all purposes.

### BACKGROUND

The introduction and temporary implantation through the skin, e.g., transcutaneously, percutaneously and/or subcutaneously, of medical devices has become very common in the treatment and/or diagnosis of patients inflicted with or suffering from any one of many different types of conditions. These implantable medical devices include those for the infusion of therapeutic or diagnostic agents, such as an infusion cannula, as well as those for monitoring a given parameter, such as a sensor, that indicates a certain bodily condition, e.g., a patient's glucose level, or the actual state of a treatment, e.g., monitoring the concentration of a drug dispensed to the patient or a body substance influenced by the drug.

In recent years, a variety of temporarily implantable sensors have been developed for a range of medical applications for detecting and/or quantifying specific agent(s), e.g., analytes, in a patient's body fluid such as blood or interstitial fluid. Such analyte sensors may be fully or partially implanted below the epidermis in a blood vessel or in the subcutaneous tissue of a patient for direct contact with blood or other extra-cellular fluid, such as interstitial fluid, wherein such sensors can be used to obtain periodic and/or continuous analyte readings over a period of time. Certain transcutaneous analyte sensors have an electrochemical configuration in which the implantable portion of these sensors includes exposed electrodes and chemistry that react with a target analyte. At an externally located proximal end of the sensor are exposed conductive contacts for electrical connection with a sensor control unit which is typically mountable on the skin of the patient. One common application of such analyte sensors systems is in the monitoring of glucose levels in diabetic patients. Such readings can be especially useful in monitoring and/or adjusting a treatment regimen which may include the regular and/or emergent administration of insulin to the patient. Examples of such sensors and associated analyte monitoring systems can be found in U.S. Pat. Nos. 6,134,461; 6,175,752; 6,284,478; 6,560,471; 6,579,690; 6,746,582; 6,932,892; 7,299,082; 7,381,184; 7,618,369 and 7,697,967; and U.S. Patent Publication Nos. 2008/0161666, 2009/0054748, now U.S. Pat. No. 7,885,698, 2009/0247857, now U.S. Pat. No. 8,346,335, and 2010/0081909, now U.S. Pat. No. 8,219,173, the disclosures of each of which are incorporated by reference herein.

These sensor devices may be designed to be positioned manually, e.g., by a user or a healthcare worker, with or without the use of an insertion device, and/or automatically or semi-automatically with the aid of a sensor insertion device. Some of these insertion devices include an introducer needle or cannula having a slotted or hollow configuration in which a distal portion of the sensor is slidably carried to the desired implantation site, e.g., subcutaneous site, after which the insertion needle can be slidably withdrawn from the implanted sensor. Examples of such insertion devices are disclosed in U.S. Pat. No. 7,381,184.

The subcutaneous or other placement of such sensors, or any medical device, produces both short-term and longer-term biochemical and cellular responses which may lead to the development of a foreign body capsule around the implant and, consequently, may reduce the flux of analyte to the sensor, i.e., may reduce the sensitivity or accuracy of the sensor function. Although many of these sensor systems are intended to be implanted over a relatively short period of time, e.g., 3-10 days, the biochemical and cellular responses begin immediately upon insertion and may have a profound and varying effect on glucose transport, often requiring numerous calibrations over the course of the sensor's implantation period. Besides the technical aspects of recalibration and the burden upon the patient to recalibrate an implantable sensor, placing the burden of calibration in the hands of patients presents safety and accuracy issues.

The extent of the immune response presented by implantable sensors, and the resulting sensor calibration and performance issues, are exacerbated by the size of the implantable portion of the sensor, often referred to as the sensor tail, and/or by the sensor introducer. A relatively large sensor tail and/or introducer outer diameter results in a more traumatic introduction which, in turn, produces a greater immune response to the sensor, as well as increased pain and discomfort felt by the patient. Accordingly, an objective of sensor manufacturers has been to minimize sensor and introducer size while providing a highly reliable and reproducible product. Such sensor miniaturization, however, requires extremely precise fabrication processes and equipment which increase manufacturing costs. For example, modifying an introducer needle or sharp, e.g., creating the longitudinal slot or slit within it, to allow it to accept a sensor requires use of very expensive laser equipment. Reducing introducer size necessarily requires reducing sensor size which, without precision fabrication and the use of highly expensive materials, will sacrifice sensor quality and reliability. Because the surface area of the electrodes or conductive traces on these miniaturized sensors is so limited, the conductive material itself must be super conductive and highly reliable, which is why many currently available implantable sensors are made with gold or platinum conductive traces, further adding to the cost of these sensors and their associated monitoring systems.

Accordingly, it would be highly desirable to provide a sensor design and associated sensor introducer, and their combined assembly, which are sized and configured to minimize trauma, pain and the immune response to sensor insertion/implantation without sacrificing sensor performance, accuracy and reliability, and which are also relatively inexpensive to manufacture.

### SUMMARY

Embodiments of implantable medical devices and methods and devices for positioning at least a portion of the medical devices beneath the epidermal layer of skin, e.g., transcutaneously, are described. A portion or the entirety of the medical devices may be implanted in a blood vessel, subcutaneous tissue, or other suitable body location. Embodiments of the implantable medical devices may provide therapeutic and/or diagnostic functions, such as the delivery of an agent to within the body or the withdrawal of a bodily fluid, or may be used for the continuous and/or automatic in vivo monitoring of the level of a bodily parameter. In certain embodiments, the implantable medical device is an in vivo analyte sensor for the continuous and/or automatic detection and measurement of one or more selected analytes.

The subject implantable medical devices as well as the devices for inserting them transcutaneously have very low profiles and dimensions to reduce the pain experienced by the patient and to reduce the traumatic effect on the tissue, thereby reducing the immune response to the insertion process and subsequent subcutaneous residence of the medical device. Furthermore, the implantable medical devices and their insertion devices are each complimentarily configured to be removably coupled together in a manner that enables a reduced profile while maximizing the functional surface area of the implantable device, and thereby optimizing device performance, accuracy and reliability.

Embodiments of the present disclosure include implantable sensors and sensor introducers having complimentary configurations and an operative assembly which provide a minimal combined cross-sectional dimension. In particular, the introducer is configured to carry a sensor about its outer surface rather than inside a slit or slot within its core structure. This configuration reduces the necessary introducer size. Moreover, a unique manner of coupling the sensor to the introducer does not sacrifice the functional surface area of the sensor, and in certain embodiments allows for an increased functional surface area, thereby optimizing sensor performance, accuracy and reliability. In certain embodiments, the increased functional surface area of the sensor enables the number of sensing elements provided on a sensor to be maximized.

Embodiments of the subject medical devices have implantable portions which have tubular constructs having cross-sectional shapes and dimensions to provide a frictionally slidable arrangement with a cylindrical introducer, such as a needle or sharp, when operatively positioned within the lumen of the tubular structure. The cross-sectional shape of the tubular structures of the implantable devices (and the corresponding cross-sectional shape of the introducers used to implant them) may have any shape including, but not limited to, circular, oval, non-circular, square, rectangular, etc., but may optimally have a configuration which minimizes cross-sectional surface area, thereby minimizing tissue trauma and pain, while maximizing the external or outer surface area of the implant, thereby optimizing functionality and performance of the device.

In certain embodiments of the subject medical devices, the non-implantable portion(s) of the device may have a non-tubular configuration, such as a substantially planar configuration, but may have any suitable construct for coupling with another component, such as a skin-mounted control unit. The non-implantable portion of the subject devices may comprise more than one section or sub-portion, e.g., a portion positioned proximally of the implantable distal portion and an intermediate portion extending between the non-implantable proximal portion and the implantable distal portion which may have a construct, e.g., that is flexible, bendable, conformable, etc., which facilitates the relative positioning of the non-implantable proximal portion with the implantable distal portion of the device.

The tubular construct of the implantable portions of the subject devices may be formed by any suitable means given the overall construct of the device. In certain embodiments, based on the necessary non-tubular construct of the non-implantable portion of devices and/or the limitations of fabricating the devices in a tubular form or only a portion of the device in a tubular form, the subject devices, including the implantable portions, have an initial, non-operative planar constructs. For example, certain of the subject devices are in vivo electrochemical sensors for measuring or monitoring a physiological or biological aspect of the body, such as analytes or the like, whereby the electrochemical elements of the sensors, i.e., the electrodes and chemical sensing components, are most easily, efficiently and/or economically fabricated on substrate material provided in an initial planar form. As such, the final, operative tubular construct of the implantable portion of the sensor is required to be formed from the initial, non-operative planar construct. This may be accomplished by various processes of the present disclosure which include folding, wrapping or winding a portion of the flat or planar construct into the desired tubular shape.

Alternatively, the entirety of the device may be provided in a tubular form and then modified in part to provide a planar portion. Still yet, the tubular and planar portions may be fabricated from separate structures, whereby the active planar structure, e.g., having the electrical/chemical components, thereon, is coupled to an inactive tubular structure, e.g., a tubular sheath, which provides only a mechanical means for being carried by an introducer for transcutaneous insertion.

These and other objects, advantages, and features of the present disclosure will become apparent to those persons skilled in the art upon reading the details of the present disclosure as more fully described below.

### BRIEF DESCRIPTION OF THE DRAWINGS

The present disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIGS. 1A and 1B illustrate planar and perspective views, respectively, of an embodiment of an implantable medical device of the type which is implantable with the insertion devices and methods of the present disclosure;

FIG. 2 is a perspective view of an embodiment of an insertion device of the present disclosure;

FIG. 3A is an isometric view of the medical device of FIGS. 1A and 1B operatively coupled to the insertion device of FIG. 2, collectively positioned on an insertion needle for transcutaneous implantation; FIG. 3B is a cross-sectional view taken along lines B-B of FIG. 3A;

FIG. 4 is a plan view of a first side of another embodiment of an implantable medical device/sensor of the present disclosure;

FIGS. 5A and 5B are perspective and end views, respectively, of the medical device/sensor embodiment of FIG. 4 in a bent or angled configuration for operative engagement with an introducer or insertion device for transcutaneous insertion;

FIGS. 6A-6C are perspective, top and side views, respectively, of the medical device/sensor of FIGS. 4, 5A and 5B operatively engaged with a transcutaneous introducer of the present disclosure;

FIGS. 7A and 7B are plan and side views, respectively, of another embodiment of an implantable medical device/sensor of the present disclosure;

FIGS. 8A-8C are perspective, top and end views, respectively, of the medical device/sensor embodiment of FIGS. 7A and 7B in a bent or angled configuration for operative engagement with an introducer or insertion device for transcutaneous insertion;

FIGS. 9A-9C are perspective, top, and end views, respectively, of the medical device/sensor of FIGS. 7A, 7B and 8A-8C operatively engaged with a transcutaneous introducer of the present disclosure;

FIG. 10 is a perspective view of another embodiment of an implantable medical device of the present disclosure;

FIG. 11A is an isometric view of the medical device of FIG. 10 operatively positioned on an insertion needle for transcutaneous implantation; FIG. 11B is a cross-sectional view taken along lines B-B of FIG. 11A; and

FIGS. 12A-12F provide perspective and end views of an embodiment of an implantable medical device of the present disclosure in various stages of fabrication.

### DETAILED DESCRIPTION

Before the subject devices, systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which the present disclosure belongs. As used herein, the terms transcutaneous, subcutaneous and percutaneous and forms thereof may be used interchangeably.

All publications mentioned herein are incorporated herein by reference to disclose and describe the methods and/or materials in connection with which the publications are cited. It is understood that the present disclosure supersedes any disclosure of an incorporated publication to the extent there is a contradiction. The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Generally, the present disclosure relates to implantable medical devices, methods for fabricating the implantable medical devices, and systems and methods for positioning or inserting an implantable medical device at least partially beneath the epidermal layer of skin. The subject devices may be implantable to provide a therapeutic and/or diagnostic function and also configured to facilitate their own transcutaneous implantation.

In certain embodiments, the implantable medical devices are sensors for detecting and measuring agents within bodily fluid, with particular embodiments that include analyte sensors for the continuous and/or automatic in vivo detection and monitoring of the level of an analyte. Analytes that may be monitored by the subject sensors include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, oxygen, peroxide, prostate-specific antigen, prothrombin, RNA, thyroid stimulating hormone, and troponin. Other of the subject sensors may be configured to detect and measure the concentration of drugs or other therapeutic agents, such as, for example, antibiotics (e.g., gentamicin, vancomycin, and the like), digitoxin, digoxin, drugs of abuse, theophylline and warfarin.

Embodiments of the subject disclosure are now further described with reference to the accompanying figures and with respect to implantable, partially implantable or in vivo sensors or sensing devices, where such descriptions are in no way intended to limit the scope of the present disclosure. It is understood, however, that the embodiments of the present disclosure are applicable to any medical device in which at least a portion of the device is intended to be positioned beneath the epidermis. Furthermore, while the implantable medical devices described in this detailed description have planar and/or tubular configurations, such shapes and descriptions thereof are not intended to be limiting, as the medical devices may have any suitable shape, including non-planar and non-tubular configurations, or may have a wire configuration.

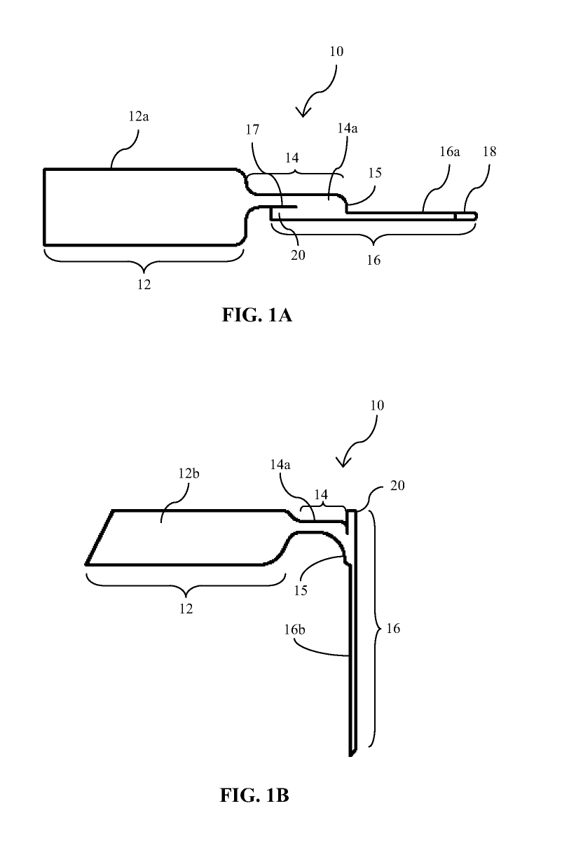

Referring now to the figures and to FIGS. 1A and 1B in particular, there is illustrated an analyte sensor 10 which in certain embodiments is configured for implantation through the surface of the skin of a patient. Sensor 10 may be described as having a proximal portion 12, an intermediate or bridging section or portion 14 and a distal portion 16. At least the implantable portion of the substrate, i.e., distal portion 16 or a sub-portion thereof, may be flexible (although rigid sensors may also be used for implantable sensors) to reduce pain to the patient and damage to the tissue caused by the insertion and/or extended implantation, i.e., “wearing”, of the sensor. A flexible substrate often increases the patient's comfort and allows for a wider range of activities. Suitable materials for a flexible substrate include, for example, non-conducting plastic or polymeric materials and other non-conducting, flexible, deformable materials. Examples of useful plastic or polymeric materials include thermoplastics such as polycarbonates, polyesters (e.g., Mylar and polyethylene terephthalate (PET)), polyvinyl chloride (PVC), polyurethanes, polyethers, polyamides, polyimides, or copolymers of these thermoplastics, such as PETG (glycol-modified polyethylene terephthalate).

Referring again to FIGS. 1A and 1B, provided on distal portion 16 of sensor 10 is a sensing element 18 including a sensing material and at least one working electrode configured to detect one or more selected analytes. Sensing element 18 may be based on “enzyme electrode” technology in which an enzyme, such as glucose oxidase or glucose dehydrogenase where the selected analyte is glucose, to provide an electrochemical enzymatic reaction when in contact with biological fluid. A detailed description of such enzymatic electrode technology is provided in for example, U.S. Pat. Nos. 6,134,461; 6,175,752; and 6,284,478, which are herein incorporated by reference.

Sensor 10 may also include one or more optional components, such as, for example, one or more additional working electrodes, a reference electrode, a counter electrode and/or a counter/reference electrode, and a temperature probe. The one or more sensor electrodes extend from sensing element 18 to the proximal portion 12 of sensor 10, over one side, i.e., an active side, of the sensor 10, including surfaces 12a, 14a and 16a, where they terminate in respective electrical contacts for coupling to corresponding electrical contacts of a sensor control/data processing unit (not shown) of an analyte monitoring system. While only a single sensing element 18 is illustrated at a very distal end of distal portion 16, any suitable number of sensing elements may be provided at any location along the length of the implantable portion of distal portion 16.

The subject sensors may be provided as part of an analyte monitoring system which includes a sensor control/data processing unit (not shown) having a housing adapted for placement on the skin surface and for coupling with the sensor electrode(s) on the proximal portion 12 of sensor 10. Communication electronics may also be disposed within the housing for relaying or providing data obtained using the sensor to another device such as a remotely located device, e.g., RF transmitter or RFID electronics. The control/data processing unit may also include a variety of optional components, such as, for example, adhesive for adhering to the skin, a mounting unit (which may include adhesive), a receiver, a processing circuit, a power supply (e.g., a battery), an alarm system, a data storage unit, a watchdog circuit, and a measurement circuit. The analyte monitoring system may also include a display unit provided on the on-skin control/data processing unit or on a separate unit remote from the on-skin unit which includes a receiver for receiving data transmitted from the on-skin unit. The remote unit may optionally include a variety of components, such as, for example, a user input mechanism, e.g., keypad, etc., a receiver, transceiver or transmitter, an analyzer, a data storage unit, a watchdog circuit, an input device, a power supply, a clock, a lamp, a pager, a telephone interface, a computer interface, an alarm or alarm system, and a calibration unit. Additionally, the analyte monitoring system or a component thereof may optionally include a processor capable of determining a drug or treatment protocol and/or a drug delivery system. Examples of such analyte monitoring systems are provided in U.S. Pat. Nos. 6,175,752; 6,284,478; 6,134,461; 6,560,471; 6,746,582; 6,579,690; 6,932,892; and 7,299,082; incorporated by reference herein.

Referring again to FIGS. 1A and 1B, the intermediate or bridging section 14 of sensor 10 may be flexible or bendable (as it would be with sensor embodiments made of the materials listed above) to allow for selectively positioning the proximal portion 12 relative to distal portion 16 to allow for a lower profile configuration above the skin surface. Alternatively, proximal portion 12 may otherwise be formed or provided at a fixed angle relative to distal portion 16, for example, if sensor 10 is made of more rigid materials such as ceramic, or the like, or connected or coupled to another component such as, for example, electronics components (printed circuit board, etc.). In either case, the proximal portion 12 may be positioned or may be positionable at an angle relative to distal portion 16 in the range from about 30° to about 180°, and more typically in the range from about 80° to about 150°. The subject sensors may be configured to be folded or bent in any suitable direction to accommodate the corresponding construct of the system components with which the proximal portion is to be coupled. For example, in the embodiment of FIG. 1B, intermediate portion 14 is flexed to provide proximal portion 12 at approximately a 90° angle to distal portion 16 such that the proximal surface 12a and distal surface 16a of a first, front or active side of sensor 10, i.e., the side of the sensor on which the sensing components are provided, are substantially inwardly facing. Alternatively, intermediate portion 14 may be flexed in the opposite direction such that proximal surface 12b and distal surface 16b of a second, back or inactive side of sensor 10 are substantially inwardly facing, where “inactive” means that no sensing components are provided thereon. In either case, the corresponding electrical contacts of the analyte monitoring control unit with which the proximal portion 12 is to be coupled must be positioned and configured to operatively couple with the sensor contacts on active proximal surface 12a, which may require proximal side 12a to face downward or toward the skin surface, as in FIG. 1B, or upward or away from the skin surface, as the case may be.

Sensor 10 may be further designed such that the central or median longitudinal axis of distal portion 16 may be disposed in a different plane in comparison to the central or median longitudinal axis of the intermediate portion and/or proximal portion 12. That is, certain embodiments of the sensor 10 may include the central longitudinal axis of distal portion 16 aligned substantially in parallel with the central longitudinal axis of the intermediate portion 14 and/or proximal portion 12 in a first geometric plane. In this manner, certain embodiments of the sensor 10 may be configured to more optimally accommodate the axis of an insertion device (not shown), discussed in greater detail below with respect to FIG. 3A. In the illustrated embodiment of FIGS. 1A and 1B, this is accomplished by a jog or shoulder 15 in the sensor substrate material between or about the juncture of the intermediate portion 14 and distal portion 16. Additionally, to facilitate alignment and/or operative coupling of distal portion 16 with an insertion device, a longitudinal cut or opening 17 may be provided within intermediate portion 14 (see FIG. 1A) to provide for a proximal tip portion 20 extending axially from distal portion 16 when sensor 10 is in a flexed, bent, curved, stressed, articulated or angled state or condition, as shown in FIG. 1B. The design or shape of the juncture between intermediate portion 14 and distal portion 16 described above in certain embodiments allows for such ambidextrous angling of the sensor.

Sensor 10, as well as the planar portions of other sensors disclosed herein, may be fabricated from well-known processes, either individually or in batches using web-based manufacturing techniques which are disclosed in U.S. Pat. No. 6,103,033, the disclosure of which is incorporated by reference in its entirety. With the latter, a continuous film or web of substrate material is provided and heat treated as necessary. The web may have precuts or perforations defining the individual sensor precursors. The various sensing elements and corresponding electrodes are then formed on the substrate web by one or more of a variety of techniques including, for example, by means of an ink jet printing process, a high precision pump and/or footed needle. Additional description of using a high precision pump with a footed needle can be found in U.S. patent application Ser. No. 12/752,109, the disclosure of which is incorporated herein by reference for all purposes. The respective material layers may be provided over a webbing of sequentially aligned sensor precursors prior to singulation of the sensors or over a plurality of sensors/electrodes where the sensors have been singulated from each other prior to provision of the one or more material layers. Next, any suitable subtractive process may be employed to remove portions of the material layers to obtain the desired size and construct of the sensing elements and electrodes. One such process includes using a laser to ablate away or trim the targeted material. After forming the individual sensing elements and electrodes, the sensor precursors, i.e., the template of substrate material (and the conductive and sensing materials), may be singulated from each other using any convenient cutting or separation protocol, including slitting, shearing, punching, laser singulation, etc. The just-described fabrication techniques are especially suitable for implantable sensors and other medical devices having completely flat or planar constructs, such as the sensor 10 of FIGS. 1A and 1B.

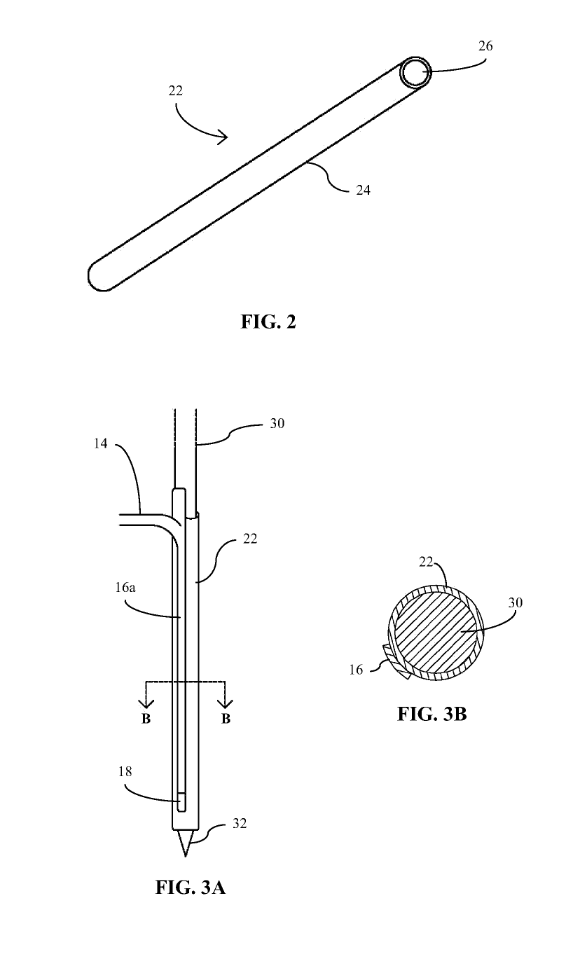

Referring now to FIG. 2, there is shown one embodiment of a medical device introducer or insertion mechanism in a sheath configuration 22 of the present disclosure for inserting a medical device, such as analyte sensor 10, transcutaneously into a patient. Sheath 22 is configured to be collectively implanted, or at least partially implanted, with the medical device for which it facilitates implantation and, subsequently, remain with and be simultaneously removed with the device, e.g., after the sensor's useful sensing life. As such, it may be made of the same material as the medical device to be implanted and have the same flexibility/rigidity/articulation as that of the device in certain embodiments. For example, sheath 22 may be fabricated from the same substrate material as sensor 10, e.g., being formed by thin film tubing techniques. Alternatively, sheath 22 may be made of a material having a greater rigidity than the medical device it is designed to implant in order to facilitate insertion into and removal from the skin.

In certain embodiments, inserter or introducer sheath 22 has an elongated configuration having an exterior surface 24 configured for fixedly engaging a surface of the medical device to be inserted and having an interior lumen 26 having a shape and dimension to accommodate an introducer, such as introducer needle or insertion needle 30 illustrated in FIG. 3A. Inserter 22 may have any suitable exterior and interior configuration and dimensions to accommodate the medical device and introducer, respectively. For example, inserter 22 may have a tubular configuration having an exterior surface 24 having a length and circumferential diameter sufficient to engage with, support and carry the distal portion 16 of sensor 10, as illustrated in FIG. 3A, but may have any exterior surface configuration to accommodate the medical device to be implanted. For example, inserter sheath 22 may have an exterior surface 24 that has a cylindrical configuration, as illustrated. With sensor portion 16 being relatively flexible, it can be deformed to fit about the rounded exterior surface of sheath 22. In embodiments where sensor portion 16 is less flexible, portion 16 may be formed with a cross-sectional shape substantially matching the exterior cross-section of sheath 22. For example, sensor portion 16 may have an arcuate cross-section having a radius of curvature which matches, complements, or correlates with that of the arcuate exterior of sheath 22. Alternatively, sheath 22 may have a flat exterior surface for carrying the medical device where the remainder of the exterior surface is annular or cylindrical to facilitate atraumatic insertion into the skin. The latter embodiment may be useful where sensor 10, or at least distal portion 16, has a flat or planar design and is made of a more rigid material and not easily flexed or folded. Alternatively, the exterior cross-section of inserter sheath 22 may have a square or rectangular configuration, or any other suitable configuration. The interior cross-sectional shape 26 of inserter 22 is typically annular to accommodate a cylindrical needle, such as introducer needle 30 of FIGS. 3A and 3B, but may have any shape to accommodate that of the introducer, including, but not limited to, oval, non-circular, square, rectangular, etc. Needle 30 may be in the form of a hypodermic needle, mandrel, sharp or the like. When provided collectively, sheath 22 and needle 30 form an introducer or insertion kit, where the sheath is a single-use implantable component of the kit and the needle may be removable after implantation of the medical device.

An operative engagement of the insertion components with sensor 10 is illustrated in FIGS. 3A and 3B with needle 30 slidably engaged within the lumen of sheath 22 and the inactive or back side 16b of distal sensor portion 16 permanently affixed to exterior surface 24 of sheath 22, either by mechanical means or by medical grade adhesive. Suitable adhesives include ultraviolet curable adhesives such as cyanocrylate glue.

In order to minimize the physical trauma to the patient and minimize the tissue response to the insertion of the implantable medical device, the cross-wise and length dimensions of sheath 22 should be as small as possible but sufficient to carry the attached medical device. With respect to a transcutaneously analyte sensor 10, for example, sheath 22 may have an outer diameter in the range from about 100 μm to about 400 μm, and more typically in the range from about 200 μm to about 300 μm, and a length in the range from about 3 mm to about 15 mm. With certain embodiments, the outer diameter, width or semi-circumferential dimension of the sheath will be substantially the same as that of the medical device to be delivered. This may be the case for embodiments in which the portion of the medical device being attached to the sheath is sufficiently flexible and, thus, able to easily conform to the outer or circumferential shape of the sheath. However, if the attachable portion of the medical device is less flexible and unable to readily conform to the sheath geometry, then the sheath may have to have a larger cross-sectional or width dimension than that of the medical device. As for the length dimension, in order to provide sufficient stability for insertion, in certain embodiments sheath 22 may have a length at least as long as the portion of distal portion 16 which will be positioned beneath the skin surface, e.g., from about 4 mm to about 8 mm, but may be longer or shorter than the implantable portion of the device. The wall thickness of sheath 22 is typically in the range from about 5 μm to about 40 μm and, in certain embodiments, ranges from about 10 μm to about 30 μm, but may be thinner or thicker as appropriate. The interior dimension of lumen 26 is such to accommodate the crosswise dimension or diameter of needle 30 which, for sensors of the type discussed here, typically has a gauge from about 30 gauge to about 33 gauge, but may be smaller or larger depending on the type of medical device and the intended application. As the sensor/sheath assembly is carried on an outer surface of introducer 30, rather than within an interior space, e.g., in a longitudinal slit or lumen of the introducer, the cross-sectional dimension of the introducer 30 is minimized to achieve the objectives of minimal tissue trauma, reduced pain and optimal sensor performance.

The component assembly, as illustrated in FIGS. 3A and 3B, may be provided preassembled, i.e., with sensor 10 pre-attached to sheath 22 and needle 30 pre-inserted or pre-loaded within sheath 22, from the factory and packaged accordingly. The pre-assembled component assembly may further include an on-skin control unit or components thereof. Alternatively, needle 30 may be provided separately and positioned within the implantable components, sensor 10 and sheath 22, by the user prior to device implantation. With user-assembled embodiments that include a mechanical means (other than adhesive) for coupling the sensor 10 to sheath 22, the sensor may be provided uncoupled from sheath 22.

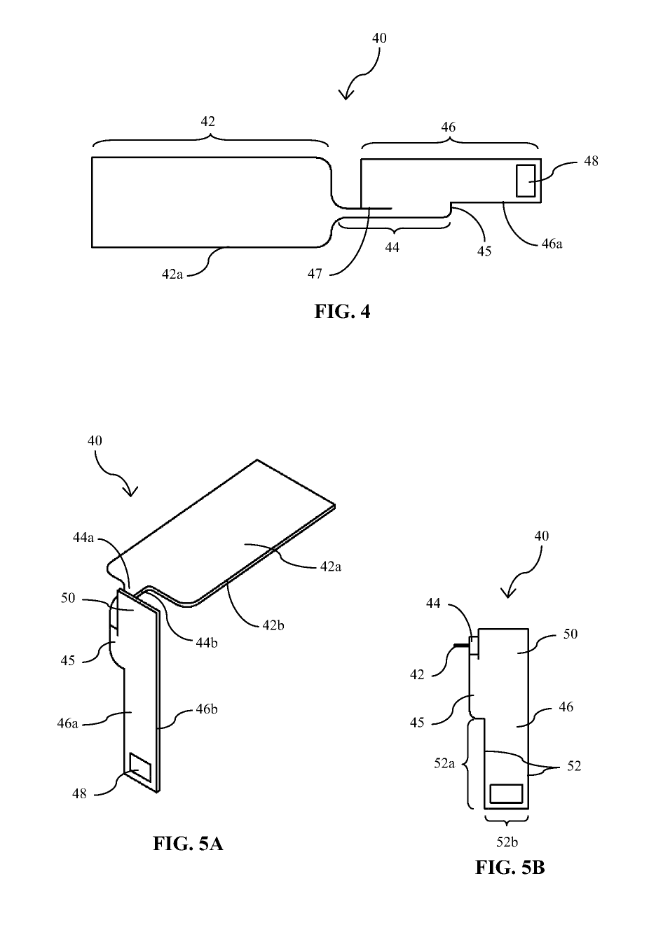

Referring now to FIG. 4, there is illustrated another embodiment of an implantable analyte sensor 40 of the present disclosure which in certain embodiments is configured for implantation through the surface of the skin of a patient. Sensor 40 has a similar structure to that of sensor 10 of FIGS. 1A and 1B, having a proximal portion 42, an intermediate or bridging section or portion 44, and a distal portion 46 having one or more sensing elements 48; however, for reasons which are discussed below, the width dimension 52b of distal portion 46 (see FIG. 5B) is substantially greater than that of distal portion 16 of sensor 10. One or more sensor electrodes (not shown) extend from sensing element 48 to the proximal portion 42 of sensor 40, over one side of the sensor, including surfaces 42a, 44a and 46a, where they terminate in respective electrical contacts for coupling to corresponding electrical contacts of a sensor control unit (not shown) of an analyte monitoring system. Further, like sensor 10, the intermediate or bridging section 44 of sensor 40 may be flexible or bendable (as it would be with sensor embodiments made of the materials listed above) to allow for selectively positioning the proximal portion 42 relative to distal portion 46, as shown in FIGS. 5A and 5B, at a desired angle, as described above with respect to sensor 10, to allow for a lower profile configuration above the skin surface. Alternatively, proximal portion 42 may otherwise be formed or provided at a fixed angle relative to distal portion 46. In the illustrated embodiment, intermediate portion 44 has been flexed, bent or angled in a direction wherein the active sides 42a and 46a of proximal and distal portions 42 and 46, i.e., the sides of the sensor on which the sensing components are provided, are outwardly facing. However, intermediate portion 44 may be folded, bent or angled in the opposite direction wherein the respective active sides 42a, 46a are facing inwardly towards each other. Also, like sensor 10, sensor 40 may have a jog or shoulder 45 and/or a cut or slit 47 in the sensor substrate material to facilitate alignment and coupling of distal portion 46 to an insertion or introducer device.

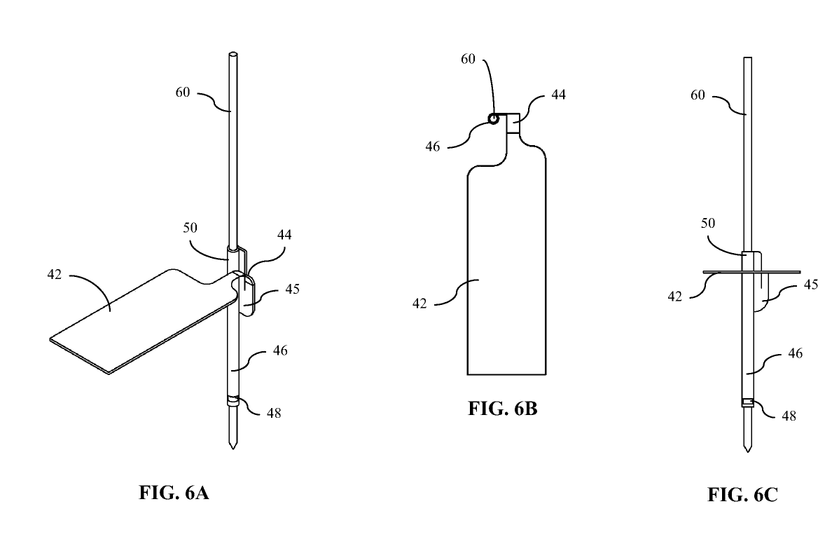

As discussed previously, the implantable medical devices of the present disclosure and their insertion devices are complimentarily configured to be coupled together in a manner that enables a reduced profile while maximizing the functional surface area of the implantable device. To this end, as illustrated in FIGS. 6A-6C, distal portion 46 of sensor 40 has been rolled or folded about its longitudinal axis in a tubular shape to provide it in a fully fabricated, operative state by which it can be operatively coupled to an introducer device 60, which is in the form of a needle or sharp. More particularly, the longitudinal edges 52 (see FIG. 5B) of distal portion 46 have been manipulated to be apposed, for example, in an edge-to-edge or overlapping arrangement, wherein the apposed edges may or may not form a longitudinal seam in the tubular portion. The active surface 46a of distal portion 46 is provided as the exterior surface when in the tubular state such that the one or more sensing elements 48 are facing outward for exposure to the subcutaneous environment and the inactive surface 46b forms the interior surface of the tubular structure having an interior diameter or crosswise dimensions for accommodating introducer 60 in a frictionally slidable engagement. Such a configuration eliminates the need for a separate insertion sheath 22. The tubular form of distal portion 46 may be provided, such as by fabrication processes described below, prior to operative engagement with introducer 60. Alternatively, in other embodiments, distal portion 46 may be wrapped about introducer 60 and fixed in the tubular format thereafter, either by a coupling mechanism (not shown), by curing or setting treatments, or by virtue of being made from a plastically deformable material.

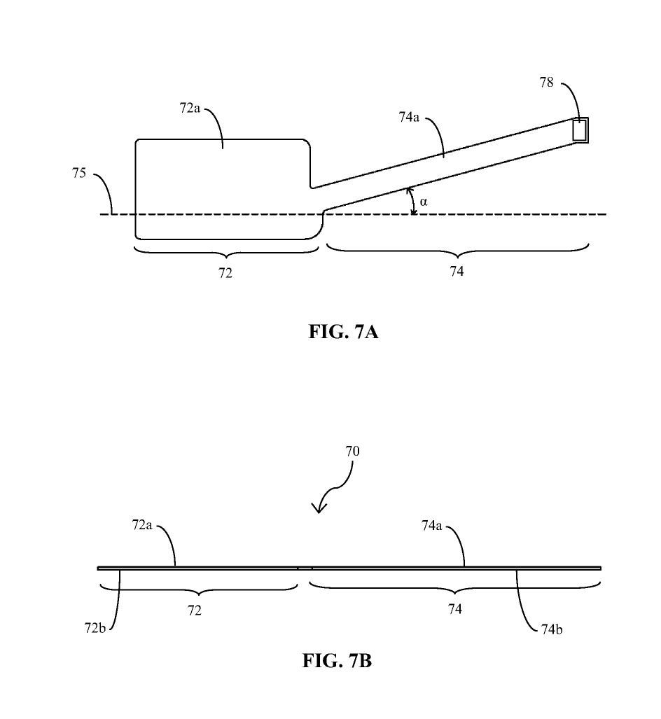

FIGS. 7A and 7B illustrate another embodiment of an implantable sensor 70 of the present disclosure in a pre-implant state in which all portions of the sensor are positioned or presented in the same plane. Sensor 70 has a proximal portion 72 which is substantially the same in structure and function to the previously described sensor embodiments. However, while distal portion 74 has a similar function to that of distal portions 16 and 46, respectively, of sensors 10 and 40, it is provided at an angle a from a major or longitudinal axis 75 of proximal portion 72, as best illustrated in FIG. 7A, where angle a is in the range from about 5° to about 30°, and more typically from about 15° to about 20°, but may be greater or smaller. As will be better understood below, this angled juxtaposition between the proximal and distal portions 72, 74 makes it unnecessary to provide an off-setting intermediate portion as described above with respect to sensor embodiments 10 and 40. While the material characteristics, e.g., flexibility/rigidity/articulation, and the overall surface area of distal portion 74 may be similar to those of distal portions 46 of sensor 40, the length and width dimensions of distal portion 74, when in the non-operative, planar configuration of FIGS. 7A and 7B, are typically longer and narrower, respectively, the purpose of which is also better understood with reference to the description below. One or more sensing elements 78, having similar electrochemical features and structures to those of the previously described sensor embodiments, are provided on first, front or active surface 74a of distal portion 74. One or more electrodes (not shown) extend from sensing element(s) 78 to proximal portion 72 and over proximal and distal active surfaces 72a, 74a. The second, back or inactive side of sensor 70 provides inactive proximal and distal surfaces 72b, 74b.

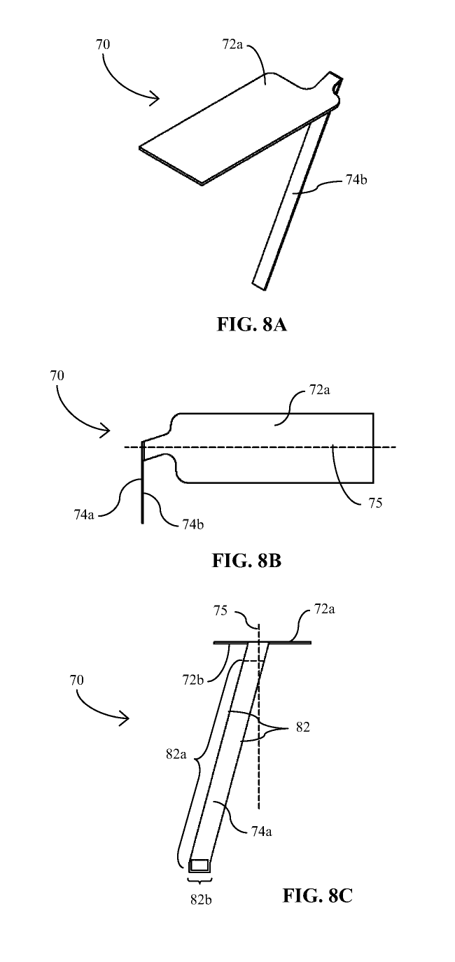

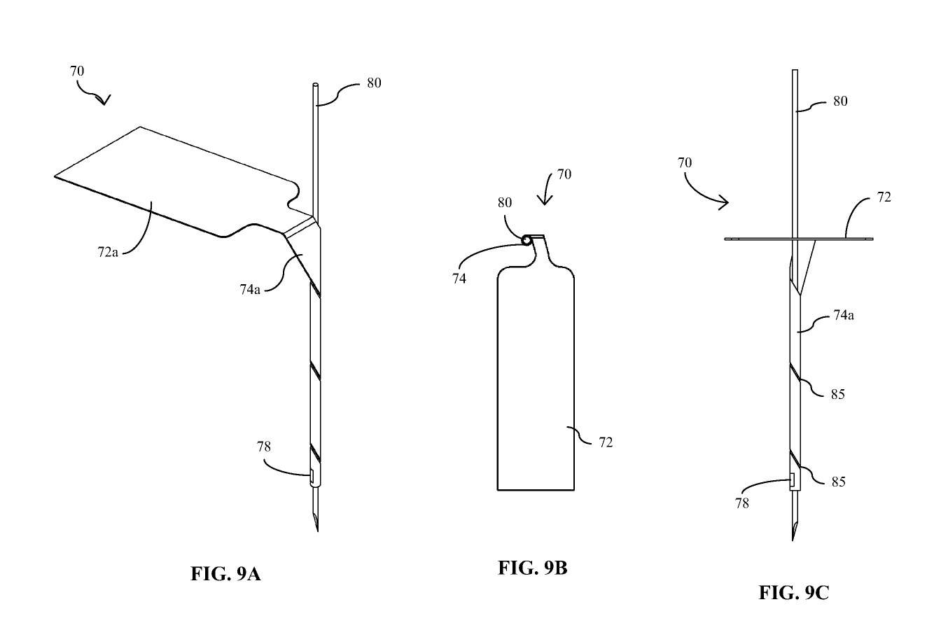

FIGS. 8A-8C show sensor 70 provided in a flexed, bent, curved, stressed, articulated or angled state or condition in which proximal portion 72 is provided at approximately a 90° angle to distal portion 74 such that active proximal surface 72a and active distal surface 74a are outwardly facing. Alternatively, sensor 70 may be flexed or bent in the opposite direction such that the opposing inactive proximal and distal surfaces 72b, 74b on the back side of sensor 70 are inwardly facing. As with the other sensor embodiments, proximal and distal portions 72, 74 may be flexed, bent, curved, stressed, articulated or angled at any suitable angle and in either direction to provide a coupling profile with the corresponding electrical contacts of an analyte monitoring control/data processing unit that is acceptable. As best observed in FIGS. 8B and 8C, the angular juxtaposition between the proximal and distal portions 72, 74, as explained above, laterally displaces distal portion 74 from the longitudinal axis 75 of proximal portion 72 when sensor 70 is in the flexed, bent, curved, stressed, articulated or angled configuration. This displacement or offset (similar to that provided by the jog or shoulder of sensors 10 and 40) enables distal portion 74 to be wrapped or wound in a somewhat transverse manner to provide a tubular configuration for operative coupling with an insertion or introducer needle 80 as shown in FIGS. 9A-9C, while maintaining the relative perpendicular positioning of proximal portion 72. More particularly, distal portion 74 has been rolled, wrapped or wound in a direction partially transversely about its longitudinal axis, e.g., in a helical fashion. The helical wrapping of distal portion 74 may be provided in a manner to provide minimal spacing 85 (see FIG. 9C) between its windings so as to minimize the overall implantable length of the sensor and/or to provide an exterior surface that is continuous and flush in order to minimize trauma to the tissue upon insertion/implantation.

When in an operative or implantable configuration, both sensor embodiments 40 and 70 are in a tubular configuration. Because of the tubular design of distal portions 46 and 74, an insertion sheath 22 is not necessary for the transcutaneous implantation of these sensors, thereby reducing the number of components and the overall cost of the collective components. Moreover, such a configuration minimizes the cross-sectional dimension of the implantable portion of the sensors (and, thus, minimizes tissue trauma and reduces pain) while maximizing their available functional/outer/active surface area to allow for a greater number and/or size of the sensing elements on a single device/sensor. Further, the greater functional surface area of the sensors allows for sensor electrodes that need not be so miniaturized and, thus, may be made of less expensive conductive materials.

The respective tubular configurations of sensors 40, 70 may be provided by various means. In one process, the sensor is fabricated in a preliminary planar form, as in FIGS. 4 and 7A, respectively, by web-based manufacturing methods or the like described above with respect to sensor 10 of FIGS. 1A and 1B. With such processes, the sensor substrate material is made of either a conformable polymer material or a metal or metal alloy, such as stainless steel foil or Nitinol, which is provided with an insulating layer on the surface that will function as the outer surface of the sensor in order to insulate the metal substrate from the electrodes and associated conductive trances. The distal portion of the sensor is then rolled, folded, wound or wrapped, as appropriate, about a cylindrical-shaped mandrel or scaffold and then heat-set or cured to provide a final, permanent tubular form. Alternatively, the sensor substrate material, or at least that used to fabricate the respective distal portions, may have physical properties which allow it to be plastically conformable or deformable without any setting or curing treatment. With either process, it may be preferential to avoid any overlapping of or spacing between the respective longitudinal edges 52 (see FIG. 5B) and edges 82 (see FIG. 8C), respectively, of sensors 40, 70 in order to provide a very flush sensor outer surface to minimize tissue trauma upon transcutaneous insertion of the sensor. Further, such edge-to-edge precision will minimize the outer cross-sectional dimension of the resulting tubular structure. With sensors made from either of the aforementioned processes, the sensing element and/or electrodes may be provided or formed on the substrate material either before or after provision of the tubular shaping.

In yet other embodiments, the sensor or at least the distal or implantable portion thereof may be provided in an original contiguous tubular form, i.e., wherein there are no seams (seamless) or spaces in the final structure, without folding, wrapping, winding or coupling the sides or ends of a precursor planar structure. Such embodiments may be fabricated by one or more extrusion methods. For example, the sensor substrate material may be made of a polymer material which may be formed in the desired tubular shape by an extrusion process, in which case the sensing components, including the conductive materials, are formed or provided on the substrate material after extrusion. In still other embodiments, the subject sensors may be fabricated by an extrusion process in which the conductive materials, e.g., metal material forming the electrode and traces, and the non-conductive materials, e.g., dielectric material forming the substrate, are co-extruded. Examples of sensors fabricated by extrusion methods are disclosed in U.S. Publication No. 2008/0200897 and U.S. patent application Ser. Nos. 12/495,618, issued as U.S. Pat. No. 8,298,158; 12/495,696, now issued as U.S. Pat. No. 8,000,763; 12/495,709; 12/495,712, issued as U.S. Pat. No. 8,437,827; and 12/495,730; all of which are incorporated herein by reference in their entireties.

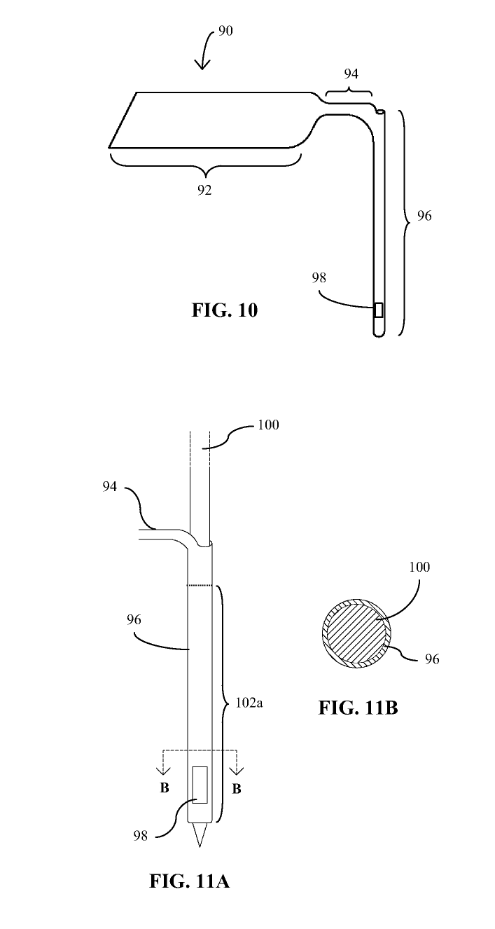

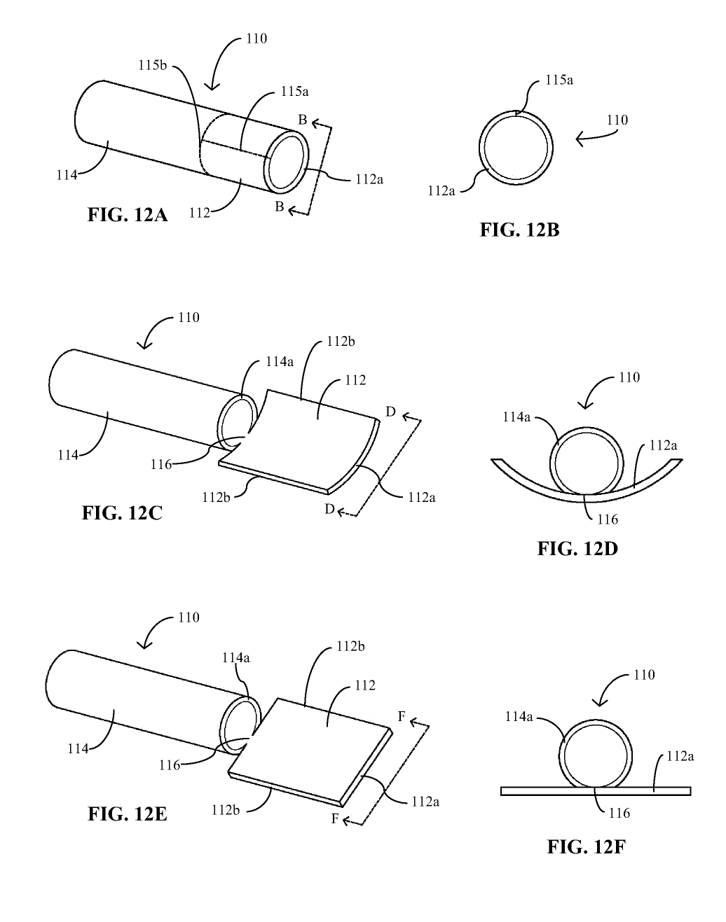

Referring now to FIG. 10, there is an embodiment of an implantable sensor 90 of the present disclosure fabricated according to an extrusion process. Sensor 90 has proximal and intermediate portions 92, 94 which are substantially the same in structure and function to those of sensors 40 and 70; however, distal portion 96 of sensor 90 is different in that it has an original configuration that is tubular or luminal rather than flat or planar. The material characteristics, e.g., flexibility, rigidity, articulation, etc., and the structural dimensions of distal portion 96 may be similar to those of the previously described sensor embodiments. A sensing element 98 is provided on an active outer surface of distal portion 96 having similar electrochemical features and structures described with respect to the sensing elements described previously. With the greater surface area that a tubular distal portion provides (rather than the strip configuration), more than one or a plurality of sensing elements (not shown) may be provided, where each sensing element may be designed to detect a particular analyte or other biological agent. Each sensing element 98 may have its own designated electrode or electrodes which extend from distal portion 96 to proximal portion 92 via intermediate portion 94. Those skilled in the art will appreciate that the construct of intermediate portion 94 may vary from that illustrated to provide a sufficient amount of surface area for bridging multiple electrodes or multiple sets of electrodes across it. As shown in FIGS. 11A and 11B, distal portion 96 of sensor 90 is operatively positioned or mounted on an insertion or introducer needle 100 having dimensions which enable slidable engagement with tubular portion 96 of the sensor.

Where the subject implantable medical devices have partial tubular constructs, i.e., only a single portion of the device is provided with an original tubular construct and the remaining portions have non-tubular constructs or are otherwise less amenable to fabrication by extrusion processes, such as with sensor 90 of FIG. 10, a hybrid fabrication approach may be taken where at least the tubular portions of these devices are formed using extrusion techniques. The other non-tubular components may be made by conventional web-based processes, such as those described above. The various components of the device may be coupled together prior to or after providing the various electrochemical components thereon, which may be formed by the deposition, printing, coating and/or removal techniques mentioned above. In other embodiments of the subject devices having both tubular and non-tubular constructs, however, the same extrusion techniques may also be used with the intended non-tubular portions of the devices, which are subsequently further processed to provide the non-tubular constructs. An example of such process is described with respect to FIGS. 12A-12F.

FIGS. 12A and 12B provide a perspective and end views, respectively, of a tubular-shaped structure or precursor 110 to an implantable medical device of the present disclosure which, in a final form, has a non-tubular or planar portion 112 and a tubular or cylindrical portion 114. For electrochemical sensor embodiments as described above, the sensor precursor is made of a substrate material(s) and has dimensions also described previously. To provide the planar portion 112, cuts 115a and 115b are made into precursor 110 using a laser or the like. Specifically, a longitudinal cut 115a is made extending from end 112a of precursor 110 to a distance within the tubular wall which defines the desired length of the side walls 112b of planar portion 112, as shown in FIG. 12C. As shown in FIG. 12A, a cross-sectional cut 115b is also made, preferably at the proximal or inside end of longitudinal cut 115a, to define an end 114a of tubular portion 114, which extends to at least about halfway or 180° through the precursor wall 110, as shown in FIGS. 12C and 12D, the latter figure being an end view of the former. Depending on the desired extent or width of the intended bridging section 116 between tubular portion 112 and planar portion 114, cross-sectional cut 115b may extend less than or more than 180° within precursor wall 110, typically to about 270° or greater. As shown in FIGS. 12C and 12D, the cut side walls 112b of planar portion 112 are then separated and folded away from each other to, in certain embodiments, a flattened condition, as illustrated in FIGS. 12E and 12F, the latter figure being an end view of the former. Optionally, planar portion 112 may then be shaped as desired and/or angled relative to tubular portion 114 by bending or folding intermediate section or bridging portion 116. The electronic components, e.g., electrodes, and electrochemical components, e.g., sensing element, etc. may be provided and formed on precursor 110 either prior to or subsequent to the cutting, shaping and bending steps just described.

The respective implantable tubular portion of each of the subject sensor devices functions as a sheath having an interior lumen which slidably and/or frictionally accommodates an introducer needle which is used to transcutaneously implant the sensor. The introducers usable with the subject sensors may be in the form of a hypodermic needle, mandrel, sharp or the like and be made of any suitable material and have an exterior surface configuration and length and diameter dimensions to sufficiently engage with, support and carry the distal portion of the sensor. As described above, for use with the subcutaneous analyte sensors of the present disclosure, the introducer typically has a gauge from about 25 to about 35, and often from about 30 to about 33, but may be greater or small to accommodate the particular medical device with which it is used. As for the length dimension, in order to provide sufficient stability for insertion, in certain embodiments, the introducer has a length at least as long as the section of the sensor's tubular portion which is intended to be positioned beneath the skin surface, but may be longer or shorter than the implantable portion of the device.

To provide an optimal ratio of cross-sectional dimension to functional surface area for the tubular distal portions of the subject sensor, their respective dimensions are as follows. For subcutaneous applications, the tubular distal portions, as well as the tubular insertion sheath such as sheath 22 of FIG. 2, typically have an implantable length dimension 52a (see FIG. 5B), 82a (see FIG. 8C) and 102a (see FIG. 11A), respectively, from about 4 mm to about 6 mm with a total length sufficient to couple with an on-skin unit. The width dimension of the sensor distal portions having precursor or pre-final configurations which are non-tubular, e.g., planar, given the typically gauge values of the introducer needles for transcutaneous insertion of in vivo analyte sensors, wherein the smaller the introducer gauge, the shorter the planar width dimension of the sensor distal portions, are as follows. The planar width dimension 52b (see FIG. 5B) of sensor distal portion 46 of sensor 40 is in the range from about 0.75 mm to about 1.75 mm, and in certain embodiments are in the range from about 0.9 mm to about 1.5 mm. For sensor distal portion 74 of sensor 70, the planar width dimension 82b (see FIG. 8C) is in the range from about 0.75 mm to about 1.25 mm, and in certain embodiments is about 1.0 mm. When the respective distal portions 46, 74 are in the operative tubular configuration (accomplished either by folding, rolling, wrapping, winding, etc. as the case may be), their resulting cross-sectional/diameter dimensions are dependent upon the wall thickness of the respective sensor distal portions and the gauge of the introducer needle. In certain embodiments, the sensor distal portions or insertion sheaths have a thickness or wall thickness in the range from about 100 μm to about 200 μm, and often between about 125 μm to about 175 μm. Of course, any of the aforementioned dimensions may be smaller or greater depending on the type of medical device being implanted and its intended application. The interior cross-sectional shape of the distal portion of the subject implantable devices when in the operative tubular form is typically annular to accommodate a conventionally shaped needle introducer, but may have any shape including, but not limited to, oval, non-circular, square, rectangular, etc., which may be formed by a corresponding shaped mandrel, scaffold or extrusion port.

The subject sensors and introducers may be provided from the factory and packaged accordingly in a pre-assembled, operative engagement with the introducer pre-inserted or pre-loaded within the sensor's distal portion. If the sensor/inserter combination is useable with an automatic insertion device or gun, the two components may be pre-assembled along with the insertion device. Alternatively, the introducer may be provided separately and operatively positioned within the distal portion of the sensor by the user just prior to device implantation.

With any of the above-described sensor embodiments, implantation of the sensor involves using the sharp tip of the introducer to penetrate the skin surface and drive the assembly to the desired depth beneath the skin surface, where insertion (and/or retraction) of the needle may be manual, automatic (where the force and speed of insertion are controlled) or semi-automatic. For example, the driving action may be provided manually by the patient or healthcare provider, where the proximal end of the introducer is equipped with a handle for operative manipulation by the user. Alternatively, an insertion gun (not illustrated) or the like may be provided having a driving mechanism for driving the introducer, the sheath and the device being carried by the sheath into the patient. The insertion mechanism may also include a retraction mechanism for removing the introducer (e.g., along the insertion path but in the opposite direction) while leaving the sensor and sheath within the patient. The driving and/or retraction functions may be fully automatic, initiated by a push of a button or the like, or semi-automatic, requiring some further manipulation by the user. Examples of such automatic or semi-automatic insertion devices are disclosed in U.S. Patent Nos. 6,175,752 and 7,381,184 and others, each of which is herein incorporated by reference.

With the subcutaneous implantation of the subject analyte sensor embodiments, the sensing element thereof is positioned under the skin so as to be in continuous contact with bodily fluid, such as blood or interstitial fluid for continuously or semi-continuously monitoring analyte levels, such as glucose levels. Of course, depending on the type of medical device, other functions may be performed by the device. The sensor is left within the skin for its useful sensing life which may be about 1 day or more, e.g., from about 3 days to about 30 days or more, e.g., about 7 days, about 10 days, about 20 days, etc.

In certain embodiments, an assembly may comprise a sensor configured for transcutaneous placement within a subject, the sensor comprising a tubular portion and a sensing element disposed on an exterior surface of the tubular portion, and a sensor introducer disposable within an interior lumen of the tubular portion of the sensor and configured to transcutaneously introduce the tubular portion through the skin of the subject.

In certain aspects, the sensor may include a planar portion extending proximally from the tubular portion, the planar portion configured for placement outside the skin of the subject for operative engagement with an external device.

In certain aspects, the planar portion may extend at an angle from the tubular portion.

In certain aspects, the angle may range from about 30° to about 180°.

In certain aspects, the sensor may include a flexible intermediate portion extending between the tubular portion and the planar portion.

In certain aspects, the tubular portion may have a proximal tip portion which extends beyond the intermediate portion when the intermediate portion is flexed.

Certain aspects may include an insertion device for driving the sensor introducer through the skin.

In certain aspects, the sensor may be an analyte sensor.

In certain aspects, the analyte sensor may be a glucose sensor.

In certain aspects, the sensor may include at least one electrode extending from the sensing element along the exterior surface of the tubular portion.

In certain aspects, the tubular portion may be formed by wrapping a planar sensor substrate into a tubular configuration.

In certain aspects, the wrapping may comprise apposing longitudinal edges of the planar substrate material.

In certain aspects, the wrapping may comprise a helical configuration.

In certain aspects, the tubular portion may be formed by an extrusion process.

In certain aspects, the tubular portion and the planar portion may be formed at least in part by an extrusion process.

In certain embodiments of the present disclosure, a system for inserting a medical device transcutaneously within a subject may comprise an introducer needle, and a sheath configured for slidable engagement about the introducer needle, wherein an exterior surface of the sheath is configured for fixed engagement with a medical device, and further wherein the sheath is configured to remain fixedly engaged with the medical device after transcutaneous insertion of the medical device.

Certain aspects may include an adhesive material for fixed engagement of the medical device with the sheath.

In certain aspects, the sheath may comprise a polymer material.

In certain aspects, the sheath may comprise one of a circular, oval or non-circular shape.

In certain aspects, the introducer needle may be part of an automated insertion device.

In certain aspects, the medical device may have a proximal portion and a distal portion, wherein only the distal portion is configured for engagement with the sheath and for transcutaneous implantation.

In certain aspects, the medical device may comprise an intermediate portion extending between the proximal portion and the distal portion, the intermediate portion being flexible to provide an angular relationship between the proximal portion and the distal portion.

In certain aspects, the medical device may comprise a sensor.

In certain aspects, the sensor may be an analyte sensor.

In certain aspects, the analyte sensor may be a glucose sensor.

In certain embodiments of the present disclosure, a method of introducing a sensor through the skin of a subject may comprise providing the sensor coupled to an exterior surface of a sheath, and using an introducer needle disposed within the sheath to transcutaneously position the sensor and the sheath through the skin of a subject.

Certain aspects may include removing the introducer needle from the subject, wherein after removal of the introducer needle from the subject, the sensor and the sheath remain transcutaneously positioned.

In certain aspects, providing the sensor coupled to the sheath may comprise adhering the sensor to the exterior surface of the sheath.

In certain embodiments of the present disclosure, an analyte sensor may comprise a tubular portion, at least a portion of which is configured for transcutaneous placement within a subject, a planar portion extending proximally from the tubular portion, and at least one electrode disposed on an outer surface of the tubular portion and on a surface of the planar portion.

In certain aspects, the planar portion may extend at an angle from the tubular portion.

In certain aspects, the angle may range from about 30° to about 180°.

Certain aspects may include an intermediate portion extending between the proximal portion and the tubular portion, the intermediate portion being bendable to provide an angular orientation between the proximal portion and the tubular portion.

In certain aspects, the tubular portion may comprise a rolled or wrapped configuration.

In certain aspects, the tubular portion may have a structure comprising no gaps or seams therein.

In certain embodiments of the present disclosure, a method of fabricating a sensor configured for at least partial implantation within a subject may comprise providing a planar substrate material having a non-implantable proximal portion and an implantable distal portion, and forming the distal portion of the substrate material into a tubular structure.

In certain aspects, the forming of the tubular distal portion may comprise wrapping the planar distal portion.

In certain aspects, wrapping the planar distal portion may comprise wrapping the planar distal portion around a cylindrical shaped mandrel.

Certain aspects may include applying heat to the cylindrically wrapped distal portion.

In certain aspects, wrapping the planar distal portion may comprise placing opposing side edges of the planar distal portion in an apposed configuration.

In certain aspects, the apposed configuration may comprise placing the side edges in an edge-to-edge configuration.

In certain aspects, the apposed configuration may comprise placing the side edges in an overlapping configuration.

In certain aspects, wrapping the planar distal portion may comprise helically winding the planar distal portion.

In certain aspects, the forming of the tubular distal portion may comprise an extrusion process.

The preceding merely illustrates the principles of the present disclosure. It will be appreciated that those skilled in the art will be able to devise various arrangements which, although not explicitly described or shown herein, embody the principles of the present disclosure and are included within its spirit and scope. Furthermore, all examples and conditional language recited herein are principally intended to aid the reader in understanding the principles of the present disclosure and the concepts contributed by the inventors to furthering the art, and are to be construed as being without limitation to such specifically recited examples and conditions. Moreover, all statements herein reciting principles, aspects, and embodiments of the present disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

1-28. (canceled)

29. A medical device comprising:
an elongate body having an arcuate exterior surface; and
a sensor body comprising a distal portion with a flat exterior surface fixedly coupled to the arcuate exterior surface of the elongate body,
wherein the distal portion comprises an electrode adapted to sense an analyte level in a human body.

30. The medical device of claim 29, wherein the sensor body comprises:
a front side where the electrode is present; and
a back side where the flat exterior surface is present.

31. The medical device of claim 30, wherein both the front side and back side of the distal portion are flat such that the distal portion has a flat design.

32. The medical device of claim 31, wherein the elongate body is an elongate tubular body.

33. The medical device of claim 32, further comprising an elongate sharp body adapted for insertion into the elongate tubular body.

34. The medical device of claim 31, wherein the elongate body is non-tubular.

35. The medical device of claim 30, wherein the distal portion comprises a plurality of electrodes.

36. The medical device of claim 29, further comprising an adhesive fixedly coupling the flat exterior surface of the distal portion to the arcuate exterior surface of the elongate body.

37. The medical device of claim 29, wherein the medical device is adapted such that it is implantable to a desired depth beneath a skin surface.

38. The medical device of claim 37, wherein the medical device is adapted such that that it is at least partially implantable beneath an epidermal layer of the skin.

39. The medical device of claim 29, wherein the elongate body has a width and the sensor body has a width, and wherein the width of the elongate body is substantially the same as the width of the sensor body.

40. The medical device of claim 29, wherein the elongate body has a width and the sensor body has a width, and wherein the width of the elongate body is larger than the width of the sensor body.

41. The medical device of claim 29, wherein the analyte level is a glucose level.
